(19) United States
(12) Patent Application Publication
(10) Pub. No.: US 2018/0292358 A1
(43) Pub. Date: Oct. 11, 2018

(54) PROBE ARRAY AND ACOUSTIC WAVE RECEPTION DEVICE

(71) Applicant: CANON KABUSHIKI KAISHA, Tokyo (JP)

(72) Inventors: Nobuhiro Ito, Yamato-shi (JP); Mitsuo Nishimura, Kawasaki-shi (JP); Hisafumi Ebisawa, Tokyo (JP)

(21) Appl. No.: 15/944,684

(22) Filed: Apr. 3, 2018

(30) Foreign Application Priority Data
Apr. 10, 2017 (JP) 2017-077674

(51) Int. Cl. G01N 29/06; G01N 29/22; G01N 29/24; G01N 29/28; A61B 5/00; A61B 8/08; A61B 8/00

(52) U.S. Cl. CPC G01N 29/0654; G01N 29/225; G01N 29/2418; G01N 29/28; A61B 8/4281; A61B 5/0095; A61B 8/0825; A61B 8/406; A61B 5/0091

(57) ABSTRACT

A probe array in which adhesion of bubbles generated in an acoustic matching liquid is reduced, and an acoustic wave reception device including the probe array are provided. The probe array includes a plurality of probes each having a reception surface which comes in contact with the acoustic matching liquid stored in a vessel, and a support portion that supports the plurality of probes and has a proximal surface which comes in contact with the acoustic matching liquid and is adjacent to the reception surface. The reception surface is equal or smaller in a contact angle with respect to the acoustic matching liquid to or than the proximal surface.

### BACKGROUND

### Field of the Disclosure

The present disclosure relates to an acoustic wave reception device including a probe array to be acoustically coupling to a subject via an acoustic matching liquid.

### Description of the Related Art

There are probe arrays including a plurality of probes for receiving acoustic waves from a subject, and an acoustic wave reception device that receives acoustic waves from a subject via an acoustic matching liquid stored in a vessel to obtain an acoustic wave image of the subject.

Japanese Patent Application Laid-Open No. 2016-55159 discusses an acoustic wave reception device that receives acoustic waves generated in a subject by irradiating the subject with a near infrared ray in order to obtain a photoacoustic image of breasts as the subject. The acoustic wave reception device discussed in Japanese Patent Application Laid-Open No. 2016-55159 includes a support base which supports an examinee and has an insertion opening, and a vessel which stores an acoustic matching liquid up to a coupling liquid level where the subject inserted from the insertion opening can be acoustically coupling to a probe array.

Japanese Patent Application Laid-Open No. 2016-55159 further discusses that the vessel is connected to a liquid supply system which can supply an acoustic matching liquid, and that bubbles in the acoustic matching liquid are reduced by adding a surface-active agent to the acoustic matching liquid.

In the acoustic wave reception device discussed in Japanese Patent Application Laid-Open No. 2016-55159, a problem is occasionally observed. The problem is that bubbles adhere to a reception surface of the probe array, receiving characteristics that vary between probes configuring the probe array, and an artifact is generated in a reconstructed acoustic wave image.

Further, downtime of the acoustic wave reception device occasionally occurs because the adhered bubbles remain on the reception surface even after a lapse of 1 minute to several hours, and thus an operating ratio of the device is decreased.

### SUMMARY

The present disclosure is directed to a probe array that reduces adhesion of bubbles to be generated in an acoustic matching liquid, and to an acoustic wave reception device including the probe array.

The acoustic wave reception device of the present disclosure includes the probe array that includes a plurality of probes each having a reception surface which comes in contact with an acoustic matching liquid stored in a vessel, and a support portion which comes in contact with the acoustic matching liquid, has a proximal surface adjacent to the reception surface and supports the plurality of probes. The reception surface is equal or smaller in a contact angle with respect to the acoustic matching liquid to or than the proximal surface.

Further features of the present disclosure will become apparent from the following description of exemplary embodiments with reference to the attached drawings.

### BRIEF DESCRIPTION OF THE DRAWINGS

FIGS. 1A and 1B are partially enlarged diagrams illustrating a vessel and a probe array according to an exemplary embodiment of the present disclosure.

FIGS. 1C and 1D are partially enlarged diagrams illustrating liquid droplet contact angles of a reception surface and of a support portion in the probe array, according to one or more embodiment of the present disclosure.

FIGS. 2A and 2B are partially enlarged diagrams illustrating contact angles of an acoustic matching liquid with respect to the reception surface and the support portion in the probe array according to an exemplary embodiment of the present disclosure.

FIGS. 2C and 2D are partially enlarged diagrams illustrating contact angles of bubbles in the acoustic matching liquid with respect to the reception surface and the support portion, according to one or more embodiment of the present disclosure.

FIGS. 3A and 3B are partially enlarged diagrams illustrating, in another probe array, the reception surface and the support portion which exhibit a function and a work of reduction in bubble adhesion in the probe array according to an exemplary embodiment of the present disclosure.

FIG. 3C is a partially enlarged diagram illustrating a vessel in the another probe array, according to one or more embodiment of the present disclosure.

FIGS. 4A and 4B are a cross section and a plan view, respectively, of an acoustic wave reception device according to an exemplary embodiment of the present disclosure.

FIGS. 5A and 5B are partially enlarged diagrams illustrating the probe array according to additional exemplary embodiments of the present disclosure.

FIGS. 6A, 6B, 6C, 6D, and 6E are partially enlarged diagrams illustrating the probe array according to yet additional exemplary embodiments of the present disclosure.

### DESCRIPTION OF THE EMBODIMENTS

Exemplary embodiments of the present disclosure will be described below with reference to the drawings.

### <Probe Array>

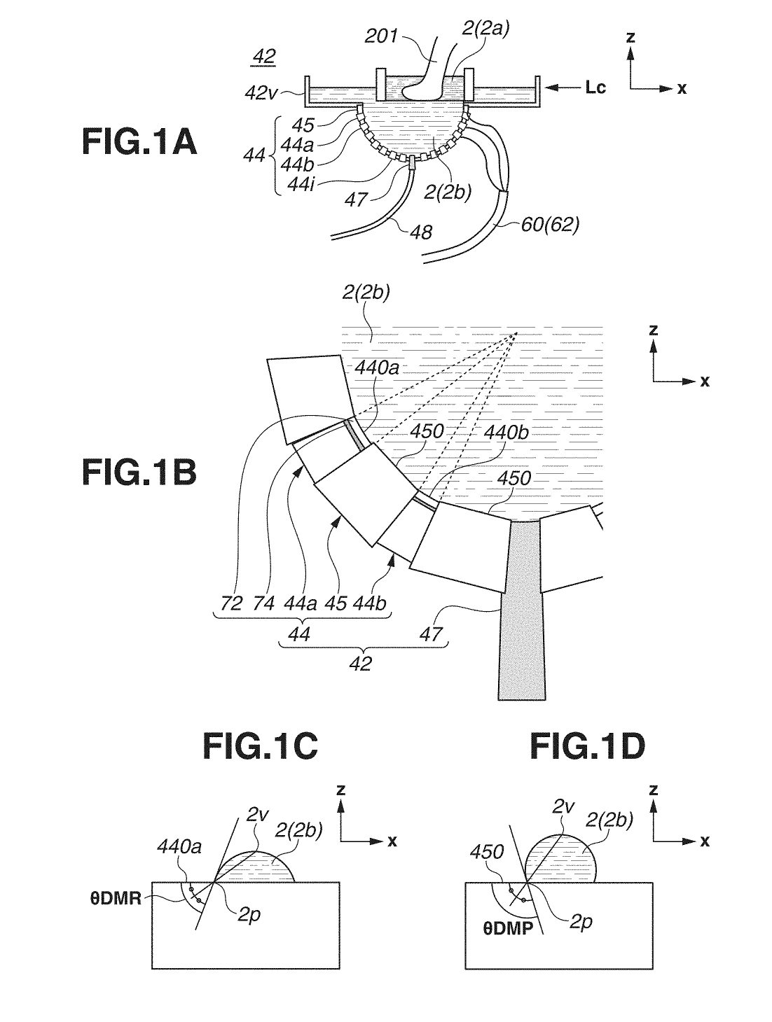

A first exemplary embodiment will be described below. A probe array that is an element of the present disclosure will be described below with reference to FIGS. 1A to 1D, FIGS. 2A to 2D, and FIGS. 3A to 3C.

FIG. 1A is a cross sectional schematic diagram illustrating a vessel 42 including a probe array 44 on a bottom of the vessel 42. FIG. 1C is a cross sectional schematic diagram illustrating a solid-liquid contact angle of an acoustic matching liquid 2 dropped onto a reception surface 440a of the probe array 44 disposed horizontally. Similarly, FIG. 1D is a cross sectional schematic diagram illustrating a solid-liquid contact angle of the acoustic matching liquid 2 dropped onto a support portion 45 of the probe array 44 disposed horizontally.

The probe array 44 according to the present exemplary embodiment includes, as illustrated in FIG. 1A, a plurality of arranged probes (44a, 44b, . . . ) and the hemispherical array support portion 45 that supports the probes. The probe array 44 is disposed on a bottom of the vessel 42. In other words, the probe array 44 configures a part of the vessel 42.

The vessel 42 includes, as illustrated in FIG. 1A, a vessel portion 42v that can store the acoustic matching liquid 2 (2b) at a coupling liquid level or more (Lc or more) at which a subject 201. The coupling liquid level indicates a lowest liquid level at which the subject 201 is in acoustically coupling to the probe array 44. In other words, the coupling liquid level indicates a lowest liquid level at which the subject is in acoustically coupling to the acoustic matching liquid 2 (2b). The vessel 42 ensures propagation of an acoustic wave from the subject 201 to the probe array 44 through the acoustic matching liquid 2 (2b).

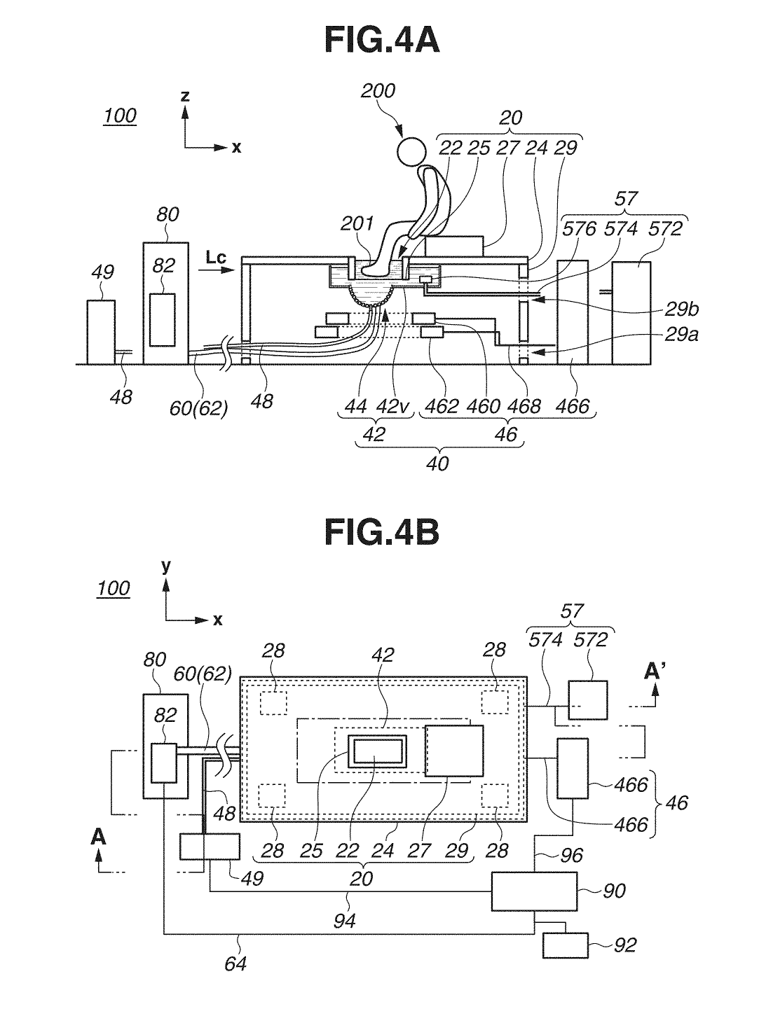

The subject 201 is held by a holding portion 25 (See FIG. 4A) which has a semi-container shape for enabling an acoustic matching liquid to be stored and is made up of a material for enabling propagation of an acoustic wave in the present exemplary embodiment. However, the subject 201 may be directly soaked in the acoustic matching liquid 2 (2b), bypassing the holding portion 25.

The probe array 44 includes, on a side in contact with the acoustic matching liquid 2 (2b), a reception surface 440 (440a, 440b, . . . ) and a proximal surface 450 adjacent to the reception surface 440. A relationship of specific surface tension is established between the reception surfaces 440a, 440b, . . . .

Probes 44a, 44b, 44i, . . . according to the present exemplary embodiment each include a not-illustrated capacitance probe capacitive micromachined ultrasonic transducer (CMUT), an acoustic matching layer 74 made of an elastic body where tungsten fine powder is dispersed, and an aluminum/alumina layer supported by the acoustic matching layer 74. The capacitance probe CMUT can be replaced by a piezo probe having another piezoelectric element.

The probes 44a, 44b, 44i, . . . are spaced from each other to output acoustic wave signals of signal strength depending on an electrode area, or each includes paired electrodes holding a piezoelectric body therebetween. Further, the reception surface 440 (440a, 440b, . . . ) is a region corresponding to an area of an electrode proximate to a side that comes in contact with the acoustic matching liquid 2 (2b) in the paired electrodes.

In the present exemplary embodiment, an aluminum/alumina layer (metal layer) 72 is anodized to a depth of 30 nm from a surface on the side in contact with the acoustic matching liquid 2 (2b) in an aluminum layer of 100 nm to be oxidized alumina which configures the reception surface 440 (440a, 440b, . . . ). The reception surface 440 (440a, 440b, . . . ) includes metal and an oxide layer of metal, and the oxide layer is disposed on the side which comes in contact with the acoustic matching liquid 2 (2b).

On the other hand, the support portion 45 is configured by coupling eight sixteenth hemispherical portions of sixteenth hemispherical aluminum in an azimuth angle direction. Each of the eight sixteenth hemispherical portions has a through hole for mounting the probe (reception surface 440 (440a, 440b, . . . )). The support portion 45 is configured so that the proximal surface 450 that comes in contact with the acoustic matching liquid 2 (2b) is anodized to a depth of 15 nm from a surface.

In the probe array 44 according to the present exemplary embodiment, since an oxidized aluminum layer is disposed on the reception surface 440 (440a, 440b, . . . ) and the proximal surface 450, both of the surfaces have hydrophilic property. However, based on a difference in a thickness of the oxidized aluminum layer, as illustrated in FIGS. 1C and 1D, the reception surface 440 (440a, 440b, . . . ) is larger in a thickness than the proximal surfaces 450, and thus has a better hydrophilic property.

The oxidized aluminum layer is called a passive layer because oxidization of the aluminum below the oxidized aluminum and an increase in the thickness of the oxide layer are suppressed by tightly covering the surface of the aluminum, and thus a layer constitution is chemically stabilized. The metal forming the passive layer may include aluminum, chrome, zinc, titanium, tantalum, niobium, zircon, combinations thereof, and derivatives therefrom, and the like.

Contact angles illustrated in FIGS. 1C and 1D are measured in the following manner. A droplet of a predetermined volume is dropped onto a targeting solid surface. At a lapse of 10 seconds after the dropping, a contact angle of a solid-liquid interface between the liquid droplet and the solid surface is measured. A ½θ method can be used for measuring the contact angle of the solid-liquid interface. The ½θ method used for measuring the contact angle of the solid-liquid interface is a measuring method for solving a problem of low measurement accuracy of a tangent defining the contact angle of the solid-liquid interface at an edge 2p formed into a circular shape around an interface between a droplet and a solid. This method is effective particularly for a solid sample with high wettability. Theory such that an angle formed between the solid surface and a segment which connects an apex 2v of the dropped liquid droplet and the edge 2p is a bisection angle of the contact angle of a solid-liquid interface is used in the measurement of the contact angle of the solid-liquid interface using the ½θ method. In FIGS. 1C and 1D, two angles each denoted by a dot correspond to a contact angle θDMR or θDMP of the solid-liquid interface.

In this specification, suffixes D, M, R, and P mean droplet (D), acoustic matching liquid (M), receive surface (R), and proximal surface (P), respectively.

In a case where the acoustic matching liquid mainly contains water, a magnitude relationship of the measurement of the contact angle of the solid-liquid interface in which the acoustic matching liquid 2 is used as the droplet can be replaced by a magnitude relationship of the measurement of a contact angle of a solid-liquid interface in which pure water (deionized water) is used as a droplet. A suffix “W” (water) is used for a contact angle θ in a case of the measurement of the contact angle using pure water as a droplet. Further, instead of the suffix D, a suffix B (bubble) is used for the contact angle θ between bubbles in a liquid, described below, and the solid surface.

In general, a contact angle of a solid-liquid interface is not a physical property value but a measurement value. In a measurement of the contact angle θ of a solid-liquid interface, errors occur in that the contact angle θ of a solid-liquid interface is excessively small in reverse proportion to the square of a diameter Φ of a droplet to be dropped and in that the contact angle θ of a solid-liquid interface is excessively small in proportion to the cube of the diameter Φ of a droplet to be dropped. The former measurement error is a reduction in volume caused by evaporation from the droplet surface, and this error affects a small-diameter droplet in which a surface area/volume becomes large. Further, the latter measurement error is deformation of a droplet edge caused by a weight of the droplet, and this error affects a large-diameter droplet in which a surface area/volume ratio becomes small.

Therefore, to minimize these errors, it is preferable to obtain a droplet size which can minimize an influence of the errors and maximize the contact angle measured apparently. In this specification, since an aqueous acoustic matching liquid with low vapor pressure is mainly used, the former influence is small. Therefore, a comparatively small diameter (is set between 0.1 mm to 0.5 mm. In general, the diameter c of a droplet to be dropped is selected from a range between 0.1 mm or more and 2 mm or less.

However, a determination whether the exemplary embodiments of the present disclosure are implementable is to measure the reception surface (the reception surface 440 (440a, 440b, . . . )) and the support portion (the proximal surface 450) under a common measurement environment, and thus does not require identification of precise physical property values.

The vessel 42 illustrated in FIGS. 1A and 1B includes an irradiation unit 47 that irradiates the subject 201 with a near infrared ray. The aluminum/alumina layer, in which the support portion 45 and the reception surface 440 (probe 44a) are disposed on a side in contact with the acoustic matching liquid 2 (2b), reflects a near infrared ray which is reflected or scattered from the subject 201 to reduce a situation that the probe array 44 becomes a noise source that generates a photoacoustic wave. The irradiation unit 47 is optically coupling to a light source (not illustrated) that generates a near infrared ray and irradiates the subject 201 with a near infrared ray. The light source continuously emits light or is controlled by a Q-switch to generate pulsed light. The light source has a light-emitting wavelength range from a visible region to a near infrared region. Therefore, its light-emitting wavelength can be in a wavelength range between, for example, 600 nm or more and 1500 nm or less.

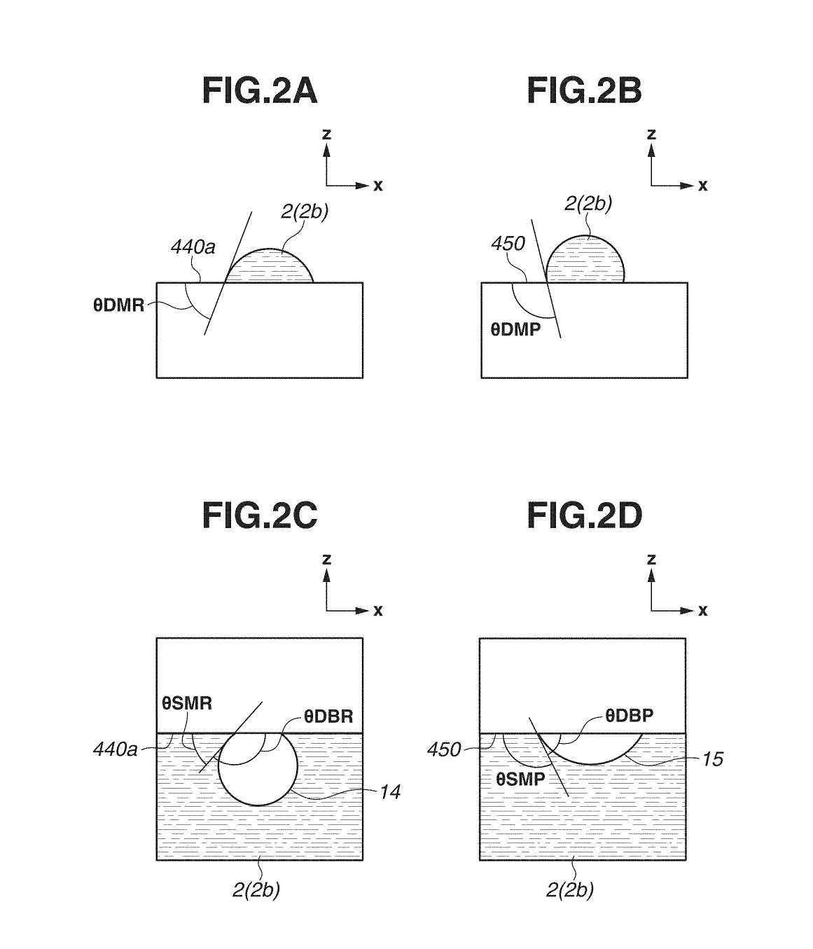

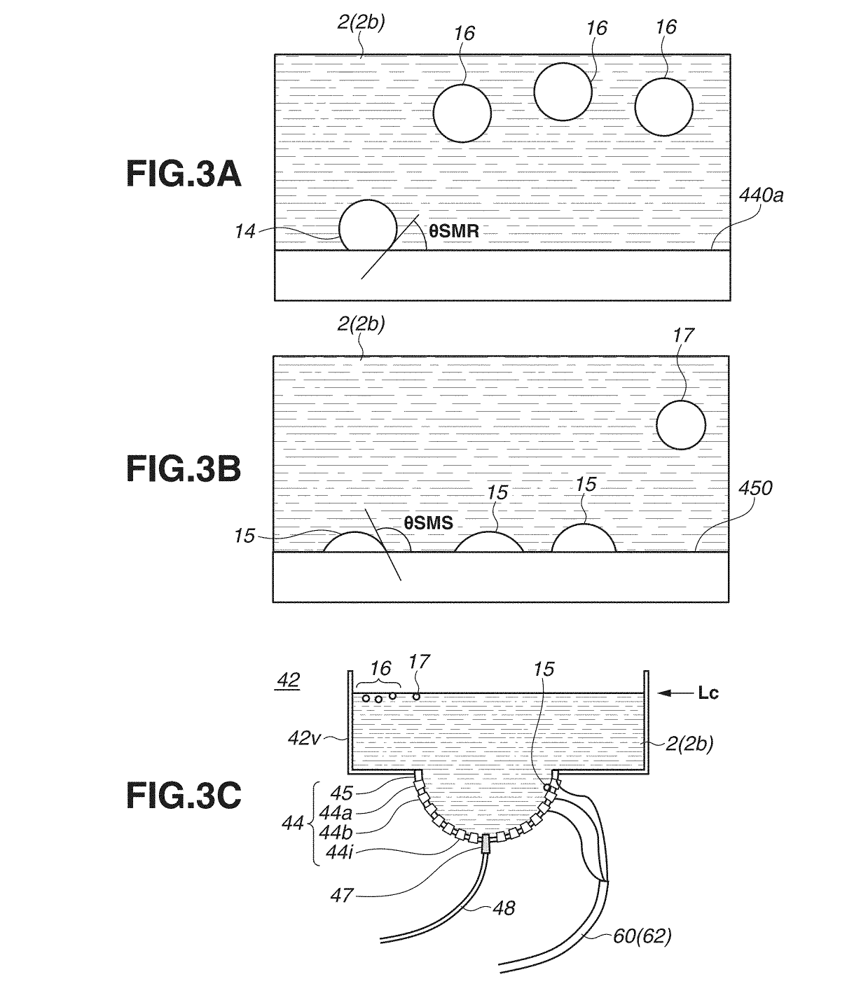

An effect in that control of the contact angle of the solid-liquid interface is linked to control of adhesion of bubbles will be described below with reference to FIGS. 2A to 2D and FIGS. 3A to 3C.

FIGS. 2A and 2B respectively illustrate the contact angles θDMR and θDMP of the solid-liquid interface in a case where the acoustic matching liquid 2 (2b) was dropped onto the reception surface 440 (440a) and the proximal surface 450 of the probe array 44 according to the first exemplary embodiment. FIGS. 2A and 2B correspond to FIGS. 1C and 1D for describing the ½θ method, respectively. Further, in FIGS. 2C and 2D, the reception surface 440 (440a) and the proximal surface 450 of the probe array 44 according to the first exemplary embodiment were placed horizontally to face down, soaked in the acoustic matching liquid 2 (2b), and states of contact with bubbles each having a diameter of about 1 mm are observed. FIGS. 2C and 2D illustrate contact angles θDBR and θDBP of a solid-gas interface.

The reception surface 440 as the solid surface illustrated in FIGS. 1A and 1C is higher in affinity with the acoustic matching liquid 2 (2b) than the proximal surface 450, and provides a surface with hydrophilic properties. As a result, the contact angle θDBP and the contact angle θDBR are expressed by the following general formula 1.

0≤contact angleθDMR contact angleθDMP general formula 1

At this time, on the proximal surface 450 has higher affinity with a bubble 15 in a liquid than with the acoustic matching liquid 2 (2b), and the contact angle θDBP of the solid-gas interface between the bubble 15 and the proximal surface 450 is smaller than the contact angle θDBR.

As a result, a contact angle θSMR and a contact angle θSMP satisfy the following general formula 2, and the contact angle θDBR and the contact angle θDBP which establish supplementary relationships with respect to the contact angle θSMR and the contact angle θSMP, respectively, are expressed by the following general formula 3.

0≤contact angleθSMR contact angleθSMP general formula 2

Contact angleθDBR≥contact angleθDBP≥0 general formula 3

From these formulas, it is understood that the magnitude relationship of the contact angles of the solid-liquid interface is negatively correlated to the magnitude relationship of the contact angles of the solid-gas interface.

Therefore, as illustrated in FIG. 3B, it is understood that a contact area with respect to the solid surface is larger than with respect to the reception surface 440 (440a), and thus the bubbles 15, which has been generated in the acoustic matching liquid 2 (2b) and has adhered to the proximal surface 450, adhere to the solid surface more stably. The bubbles 15 adhering to the proximal surface 450 are separated from the interface by a buoyancy and a fluid pressure of the acoustic matching liquid 2 (2b) and then become bubbles 17. However, the separation from the proximal surface 450 requires comparatively longer time than from the reception surface 440 (440a).

On the other hand, as illustrated in FIG. 3A, it is understood that a contact area with respect to the solid surface is smaller than with respect to the proximal surface 450, and thus bubbles 14 which have been generated in the acoustic matching liquid 2 (2b) and have adhered to the reception surface 440 (440a), adhere to the solid surface more unstably. The bubbles 15 adhering to the reception surface 440 (440a) are easily separated from the interface by a buoyancy and a fluid pressure of the acoustic matching liquid 2 (2b) and are then become bubbles 16.

It is difficult for the unstable bubbles 14 providing a high contact angle of the solid-gas interface to remain on the reception surface 440 (440a) due to flux of the acoustic matching liquid 2 (2b), a gradient of the probe array 44 and the like. The bubbles 14 thus move to a region where a propagation path of an acoustic wave received by the probe 44a is not disturbed.

A moving destination of the separated bubbles 16 is, as illustrated in FIG. 3C, the proximal surface 450. Alternatively, the bubbles 16 may be separated from the probe array 44 to rise to a coupling liquid level Lc of the acoustic matching liquid 2 (2b) due to buoyant or flux of the acoustic matching liquid 2 (2b).

Degradation of imaging quality caused by adhesion of bubbles to the reception surface and downtime to be required for separation of bubbles can be reduced by applying the probe array 44 according to the first exemplary embodiment to an acoustic wave reception device including a vessel.

The support portion 45 has a hemispherical shape in the present exemplary embodiment but may have a quadric surface of revolution such as ellipsoid of revolution, paraboloid of revolution, or hyperboloid of revolution.

### <Contact Angle, and Control of Surface Tension>

In the probe array 44 according to the present exemplary embodiment, as illustrated in FIGS. 1C and 1D, the reception surface 440 (440a) (reception surface) and the proximal surface 450 (support portion) are controlled so that the contact angle with respect to the acoustic matching liquid 2 (2b) is equal between them or is smaller on the reception surface 440 (440a) than on the proximal surface 450. In other words, the reception surface 440 (440a) is controlled so as to be equal or larger in a surface tension to or than the proximal surface 450.

Further, in other words, in a case where the acoustic matching liquid 2 (2b) mainly contains water, the reception surface 440 (440a) is equivalent or higher in hydrophilic properties to or than the proximal surface 450.

In other words, crystallographically a solid material having large surface tension has large bonding energy between their atoms. In general, a ranking of bonding, from the largest energy to the smallest energy, is covalent bonding, ion bonding, and metallic bonding. Therefore, metal alloy of carbide, oxide, and nitride of silver (Ag), copper (Cu), or aluminum (Al) provides higher surface tension than pure metal or metal alloy including Ag, Cu, or Al provides.

Further, it is said from a viewpoint of material engineering that a solid material with high surface tension has a high elastic constant and a high rigidity modulus. This corresponds to that a ceramic material or a glass material has a higher elastic constant than metal having ductility and malleability. Further, in a case where the surface includes a thin layer with a thickness of 1E-6 m, a distribution of the surface tension may be formed representatively by an elastic modulus of a support layer which supports the thin layer and is larger in a thickness than the thin layer.

Further, it is understood that the solid material with high hydrophilic properties includes a lot of hydrophilic groups such as hydrogen (H) or hydroxyl (OH) as a surface composition or includes a small number of hydrophobic groups such as hydrocarbon exhibiting hydrophobic properties.

Further, it is understood that the solid material with high hydrophilic properties includes a lot of materials which form hydrate on a surface of the solid material.

Therefore, it is preferable that the reception surface 440 (440a) has higher bonding energy than the proximal surface 450 has. Similarly, in a case where the reception surface 440 (440a) and the proximal surface 450 contain the same type of a metallic element, the reception surface 440 (440a) is made to be equal to or higher than the proximal surface 450 in surface concentration of oxide, so that the reception surface 440 (440a) exhibits higher or equal surface tension than or to the proximal surface 450.

Further, it is preferable that in a case where the reception surface 440 (440a) and the proximal surface 450 each have a thin layer with thickness of 1E-6 m or less on their surfaces, elasticity of the support material that supports the thin layer is high. In a case where the reception surface 440 (440a) and the proximal surface 450 each have the thin layer with thickness of 1E-6 m or less on their surfaces and are supported by an elastic material with lower elasticity than that of the thin layer, the reception surface 440 (440a) is caused to exhibit higher surface tension than the proximal surface 450 exhibits by thinning the thickness of the material which supports the reception surface 440 (440a).

Further, in a case where a main liquid composition of the acoustic matching liquid 2 (2b) is water, the reception surface 440 (440a) is caused to exhibit a higher or equal hydrophilic property than or to the proximal surface 450 by making surface concentration of a hydrophilic group in the reception surface 440 (440a) higher than in the proximal surface 450. Similarly, in the case where the main liquid composition of the acoustic matching liquid 2 (2b) is water, the reception surface 440 (440a) is caused to exhibit a higher or equal hydrophilic property than or to the proximal surface 450 by making surface concentration of a hydrate forming material higher in the reception surface 440 (440a) than in the proximal surface 450. Examples of the hydrate forming material include oxidized aluminum (alumina), oxidized chrome, and oxidized titanium.

Differently from the surface tension of a liquid, the surface tension of a solid surface cannot directly be determined quantitatively, but this problem is solved by a Zisman plot method. In the Zisman plot method, critical surface tension to be given by intercept of cos θ=1 (completely wet state) is determined as the surface tension of the solid surface by using a plot in which the contact angle has been measured by using different types of liquids for one kind of the targeting solid surface.

### <Acoustic Wave Reception Device>

A second exemplary embodiment will be described below. FIGS. 4A and 4B are a cross section and a plan view illustrating an acoustic wave reception device 100 according to the second exemplary embodiment which includes the probe array 44 according to the first exemplary embodiment of the present disclosure, respectively. FIG. 4A is a vertical cross section in a case where a configuration along a plane A-A′ in FIG. 4B is virtually viewed. The plane A-A′ is an anomalistically bent surface for easy understanding. FIG. 4B is the plan view in a case where the acoustic wave reception device 100 illustrated in FIG. 4A is viewed from above in the direction of a z-axis.

The acoustic wave reception device 100 according to the second exemplary embodiment includes a support base 20, the vessel 42 having the probe array 44, a two-dimensional scanning unit 46 that scans the vessel 42, and a temperature control mechanism 57 that adjusts a temperature of the acoustic matching liquid 2 (2b) stored in the vessel 42. Each of the elements will be described below.

The support base 20 includes, as illustrated in FIG. 4A, an insertion portion 22 for inserting the subject 201 as a part of an examinee 200, and a support portion 24 that supports the examinee 200.

The support base 20 according to the present exemplary embodiment further includes, as illustrated in 4A, the holding portion 25 that holds the subject 201 on a position overlapped with the insertion portion 22, a seat 27 on which the examinee 200 sits, and a side panel 29 continuous to four sides around the support portion 24 on a top panel. The support base 20 further includes, as illustrated in FIG. 4B, four columns 28 that support the support portion 24.

The seat 27 is disposed on the support portion 24 so that an imaging orientation of the examinee 200 is made to be stable. Further, the side panel 29 is provided so as to surround the vessel 42 that moves during image-capturing, an X stage 460, a Y stage 462, and the irradiation unit 47, and separate a movement space of the examinee 200 and an operator (not illustrated), from inside of the device. A cushion (not illustrated) may be disposed on the support portion 24 and the seat 27 to reduce a load of the examinee 200 during image-capturing.

The insertion portion 22 is an opening provided in the support portion 24 so as to enable the subject 201 as a part of the examinee 200 to be inserted. A portion of the examinee 200 which has not been inserted into the insertion portion 22 can be placed on the support portion 24 so that the imaging orientation of the examinee 200 can be made to be stable. The portion to be imaged includes upper limb, lower limb, a head, and breasts of the examinee 200. However, FIG. 4A illustrates an imaging orientation at a seated position when the left leg of the examinee 200 as the subject 201 is inserted into the insertion portion 22 and his/her right leg (not illustrated) is placed on a mount portion of the support portion 24.

The holding portion 25 is secured to the support portion 24 at a position overlapped with the insertion portion 22 because of an intention to stabilize the subject 201 during image-capturing. The holding portion 25 protrudes downward from the support portion 24, i.e., has a semi-container shape, and enables the subject 201 to be held below the support portion 24.

The holding portion 25 is made of an acoustic matching material having a propagation property (low attenuation property) of an acoustic wave. The propagation property enables the probe array 44 to receive an acoustic wave propagated from the subject 201. A resin material such as isoprene rubber (IR), silicone rubber, or polyethylene terephthalate for allowing transmission in an infrared region is used as the material of the holding portion 25. The exemplary embodiments of the present disclosure include also a form where the material is flexible and a mesh-type resin material having higher rigidity than that of the flexible member is used in combination for compensating a lower end of the holding position.

It is desirable that the holding portion 25 be airtight for separating spaces so that the acoustic matching liquid 2 (2b) stored in the vessel 42 does not directly contact with the subject 201. This makes it possible to ensure hygiene property for the acoustic matching liquid 2 (2b) stored in the vessel 42. The holding portion 25 being airtight and the semi-container shaped projection stores the acoustic matching liquid 2 (2a), and acoustic coupling between the holding portion 25 and the subject 201 can be secured.

A handrail or a protection fence (not illustrated) may be suitably provided on the support base 20 in order to reduce a feeling of anxiety of the examinee 200.

A reception scanning unit 40 includes the probe array 44 that receives an acoustic wave propagated from the subject 201 inserted via the insertion portion 22, and the two-dimensional scanning unit 46 that two-dimensionally moves the probe array 44 in parallel with a horizontal plane. The reception scanning unit 40 is disposed below the support portion 24.

The two-dimensional scanning unit 46 includes, as illustrated in FIGS. 4A and 4B, the X stage 460 that is disposed below the support portion 24 and can move the probe array 44 in a first direction dl. The X stage 460 according to the present exemplary embodiment moves the vessel 42, thereby moving the probe array 44 in an x direction.

The two-dimensional scanning unit 46 includes, as illustrated in FIGS. 4A and 4B, the Y stage 462 that is disposed below the X stage 460 and can move the X stage 460 in a y direction intersecting the first direction dl.

In other words, the X stage 460 and the Y stage 462 are an XY stage that relatively moves the probe array 44 with respect to the insertion portion 22 in two dimensions and in parallel with the horizontal plane. The X stage 460 and the Y stage 462 can scan, based on a scanning signal to be output from a scanning control circuit 466 (described below), the probe array 44 in any two-dimensional scanning pattern including rotational scanning, spiral scanning, Boustrophedon scanning, and raster scanning. In other words, the vessel 42 is connected to the two-dimensional scanning unit 46 that scans the probe array 44 so that a relative position with respect to the subject 201 shifts.

The two-dimensional scanning unit 46 includes the scanning control circuit 466 that outputs a scanning signal to the X stage 460 and the Y stage 462, and a scanning signal cable 468 that connects the X stage 460, the Y stage 462, and the scanning control circuit 466.

The scanning control circuit 466 is disposed outside the support base 20, and outputs a scanning signal to each of the X stage 460 and the Y stage 462 via the scanning signal cable 468 wired via a cable opening 29a provided on the side panel 29. Wireless transmission of a scanning signal can omit the scanning signal cable 468.

The “horizontal state” in the specification of the present disclosure means a physical quantity that can be observed using a level or a laser displacement meter. In the acoustic wave reception device 100 according to the present exemplary embodiment, an effective gradient allowance is present in the horizontal state, and an upper limit and a lower limit of a gradient angle tan θ are within ±0.5 mm/m with respect to a complete horizontal state which is perpendicular to a vertical direction. The upper limit and the lower limit of the gradient angle tan θ are preferably within ±0.1 mm/m, or more preferably within ±0.04 mm/m.

In the acoustic wave reception device 100 according to the present exemplary embodiment including the vessel 42 to be two-dimensionally scanned together with the probe array 44 and the acoustic matching liquid 2 (2b), flux, generation of waves, and holding of bubbles are caused by inertia in the acoustic matching liquid 2 (2b) stored in accordance with the scanning.

It is particularly preferable that the probe array, in which the affinity with the acoustic matching liquid 2 (2b) according to the present exemplary embodiment is controlled, is applied to the acoustic wave reception device 100 including the vessel 42 connected to the two-dimensional scanning unit 46.

Further, the vessel 42 according to the present exemplary embodiment is connected to a liquid supply mechanism (not illustrated). The liquid supply mechanism includes a reservoir tank, a pump, and piping, and supplies the acoustic matching liquid into the vessel 42. The liquid supply mechanism may be a sealed system which blocks air. However, such a system has a complicated structure and requires difficult maintenance. Thus, an open-type liquid supply mechanism is generally used. In the open-type liquid supply mechanism, a gas component is dissolved in the acoustic matching liquid to be supplied to the vessel 42, and thus bubbles are generated simultaneously with the supply of the liquid to the vessel 42.

It is particularly preferable that the probe array in which the affinity with the acoustic matching liquid 2 (2b) according to the present embodiment is controlled is applied to the acoustic wave reception device 100 including the vessel 42 connected to the open-type liquid supply mechanism. The liquid supply mechanism has a surface where the acoustic matching liquid comes in contact with air, and is connected to the vessel 42 to supply the acoustic matching liquid to the vessel 42.

The acoustic wave reception device 100 according to the present exemplary embodiment includes the temperature control mechanism 57 that controls a temperature of the acoustic matching liquid 2 (2b) stored in the vessel 42. The temperature control mechanism 57 is disposed in the vessel 42 and includes a heat radiation/absorption device 576 having a heater, a Peltier element, and a heat pipe, a temperature adjustment cable 574, and a temperature control circuit 572.

The temperature control mechanism 57 is provided to manage a sound speed of the acoustic matching liquid 2 (2b) within a predetermined range or reduce a difference in temperature between the acoustic matching liquid 2 and the subject 201. In the temperature-controlled acoustic matching liquid 2 (2b), dissolution of gas in a temperature drop process is improved, and bubbles originated from the dissolved gas is generated in a temperature rise process.

It is particularly preferable that the probe array, in which the affinity with the acoustic matching liquid 2 (2b) according to the present exemplary embodiment is controlled, is applied to the acoustic wave reception device 100 including the temperature control mechanism 57 according to the present exemplary embodiment.

The acoustic wave reception device according to the present exemplary embodiment further includes a plurality of signal lines 60 that transmits acoustic wave signals respectively output from the plurality of probes (44a, 44b, 44i . . . ) and a signal relay 80 that is electrically connected with the probe array 44 via the plurality of signal lines 60.

The probe array 44 according to the present exemplary embodiment outputs a received acoustic wave as an analog acoustic wave signal. Therefore, the plurality of signal lines 60 configures a parallel transmission cable 62 that has channels of which the number is equal to the number of the plurality of probes (44a, 44b, 44i) in the probe array 44 and that transmits (analog) acoustic wave signals of the respective channels in parallel. The parallel transmission cable 62 is a cable group in which some or all of the plurality of signal lines 60 are tied in a bundle.

The signal relay 80 includes an AD converter 82 that converts the analog acoustic wave signals transmitted in parallel from the probe array 44 into digital acoustic wave signals. The signal relay 80 serially transmits the converted digital acoustic wave signals to an integrated control unit 90 having a signal processing circuit (not illustrated) via a serial cable 64. The serial cable 64 is a cable for transmission of digital signals in chronological order.

Therefore, in other words, the signal relay 80 is a relay that relays the cable group (the parallel cable 62) to which analog signals are transmitted in parallel with the cable (the serial cable 64) to which digital signals are transmitted in chronological order.

The signal processing circuit of the integrated control unit 90 reconstructs the digital acoustic wave signals output from the signal relay 80 and outputs captured images to a storage medium (not illustrated) or a display unit 92. Further, the form in which the integrated control unit 90 includes the storage medium (not illustrated) is included as an exemplary embodiment in the present disclosure. The integrated control unit 90 can output a control command to the scanning control circuit 466, the liquid supply mechanism (not illustrated), and the temperature control mechanism 57.

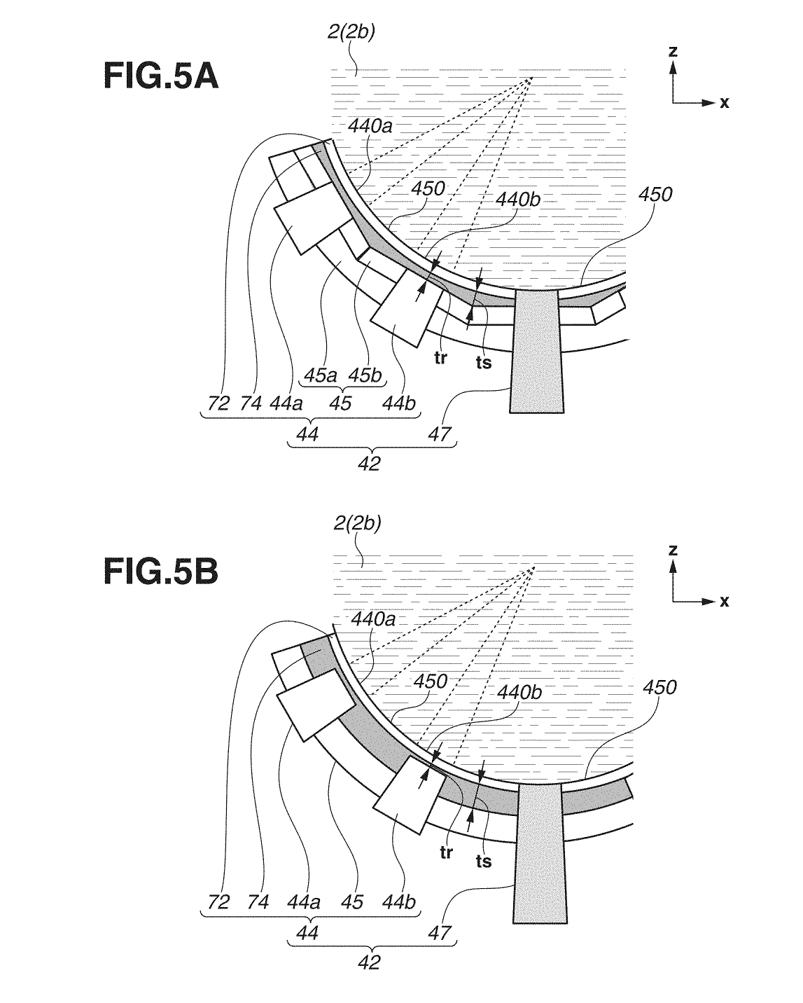

Further, the probe array 44 according to the present exemplary embodiment includes, as illustrated in FIGS. 3C and 5A, the irradiation unit 47. The irradiation unit 47 is optically connected to an optical fiber 48 which transmits a near infrared ray output from a light source 49 which outputs pulsed light in a near infrared ray region. A photoacoustic wave generated inside the subject 201 by the near infrared ray emitted from the irradiation unit 47 toward the subject 201 is received by the probe array 44.

Third and fourth exemplary embodiments will be described below. The probe array 44 according to the third and fourth exemplary embodiments illustrated in FIGS. 5A and 5B is different from the probe array 44 according to the first exemplary embodiment in that a metal layer 72 is provided to be shared by the reception surface 440 (440a, 440b, . . . ) and the proximal surface 450.

The metal layer 72 has a function for controlling affinity with the acoustic matching liquid 2 (2b) to be uniform. The probe array 44 according to the third and fourth exemplary embodiments includes the acoustic matching layer 74 that supports the metal layer 72.

The probe 44a (44b, . . . ) of the probe array 44 according to the third exemplary embodiment is mounted to the support portion 45 by a flat plate-type flange member 45a (45b, . . . ). In other words, the flange members 45a, 45b, . . . are spread all over the support portion 45.

The acoustic matching layer 74 is disposed so as to fill a gap between the hemispherical metal layer 72 and the spread flange members 45a, 45b, . . . . It is preferable that the acoustic matching layer 74 is made of an elastic body in order to reduce generation of a transverse wave. The acoustic matching layer 74 is obtained by dispersing metal fine particles such as tungsten in rubber.

In a case where a gold thin layer with thickness of 100 nm is disposed as the metal layer 72 on the side which comes in contact with the acoustic matching liquid 2 (2b), the probe array 44 according to the present exemplary embodiment exhibits a wettability distribution of a surface of the gold thin layer due to a difference in contribution of the acoustic matching layer 74 which supports the metal layer 72.

The metal layer 72, which corresponds to the reception surface 440 (440a) including the thin acoustic matching layer 74 made of the elastic body, is larger in surface tension than the metal layer 72 corresponding to the proximal surface 450 including the thin acoustic matching layer 74. Further, the metal layer 72 of the reception surface 440 (440a) has a small solid-liquid contact angle with respect to the acoustic matching liquid 2 (2b).

In other words, the metal layer 72 is a reflection layer that further reflects a near infrared ray reflected from the subject toward the subject.

The probe array 44 according to the fourth exemplary embodiment illustrated in FIG. 5B is different from the probe array 44 according to the third exemplary embodiment in that the flange members are not used and the plurality of probes 44a, 44b, 44i, . . . is disposed to protrude toward an inside of the hemispherical array support portion 45. The probe array 44 according to the present exemplary embodiment is similar to that according to the third exemplary embodiment in that the acoustic matching layer 74 is disposed to fill a gap between the hemispherical metal layer 72 and the support portion 45 or the plurality of the protruded probes 44a, 44b, 44i, . . . , and the acoustic matching layer 74 has a thickness distribution.

The probe array 44 according to the present exemplary embodiment exhibits a wettability distribution of the surface of the gold thin layer due to a difference in contribution of the acoustic matching layer 74 that supports the metal layer 72.

Increase in secondary contamination in the probe array 44 due to microbial occurrence in the probe array 44 and the vessel 42 can be reduced by using metal such as silver, copper, or gold exhibiting an antibacterial effect for the metal layer 72. The metal layer 72, which corresponds to the reception surface 440 (440a) including the thin acoustic matching layer 74 made of the elastic body, is larger in surface tension than the metal layer 72 corresponding to the proximal surface 450 including the thin acoustic matching layer 74. Further, the metal layer 72 corresponding to the reception surface 440 (440a) has a low solid-liquid contact angle with respect to the acoustic matching liquid 2 (2b).

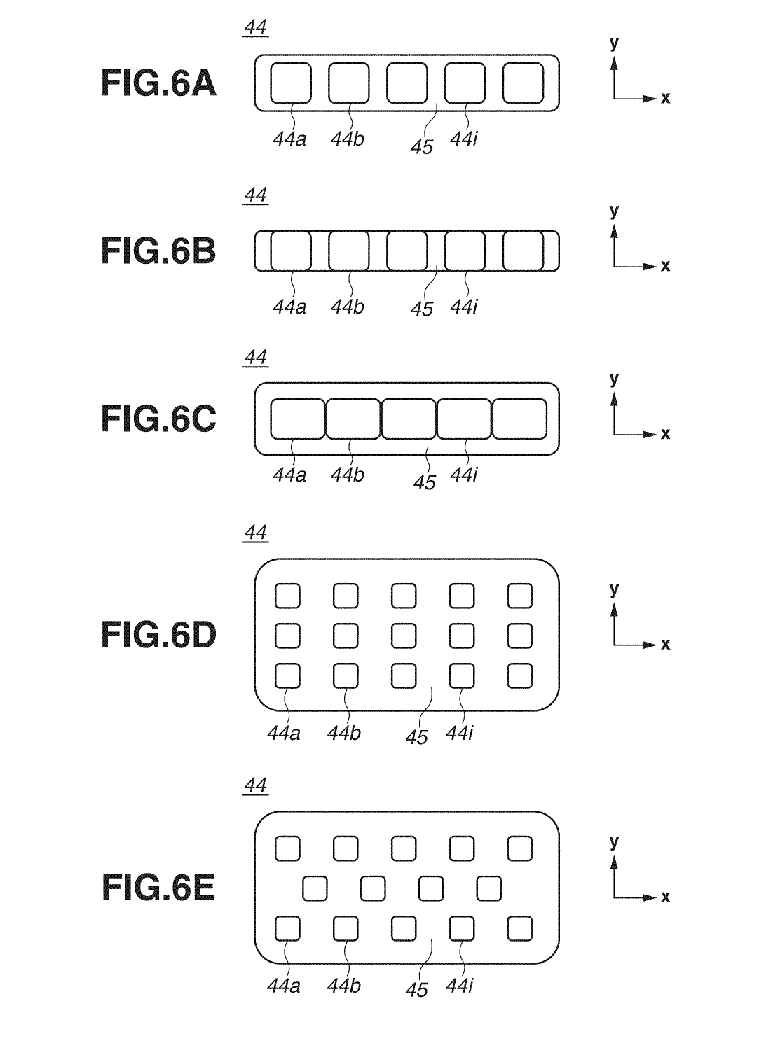

Fifth, sixth, seventh, eighth, and ninth exemplary embodiments will be described below. The probe array 44 according to the first to fourth exemplary embodiments has a hemispherical inner surface, and the plurality of probes 44a, 44b, 44i, . . . is disposed three-dimensionally. A one-dimensional array illustrated in FIGS. 6A to 6C and a two-dimensional array illustrated in FIGS. 6D and 6E are applicable to the probe array of the present disclosure.

The probe array 44 according to the fifth to ninth exemplary embodiments respectively illustrated in FIGS. 6A to 6E includes a plurality of reception surfaces (not illustrated) corresponding to the plurality of probes 44a, 44b, 44i, . . . , and a proximal surface (not illustrated) corresponding to the support portion 45 adjacent to the plurality of probes 44a, 44b, 44i, . . . .

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2017-077674, filed Apr. 10, 2017, which is hereby incorporated by reference herein in its entirety.

What is claimed is:

1. A probe array comprising:
a plurality of probes each having a reception surface which comes in contact with an acoustic matching liquid stored in a vessel; and
a support portion configured to support the plurality of probes, the support portion having a proximal surface which comes in contact with the acoustic matching liquid where the proximal surface is adjacent to the reception surface,
wherein the reception surface is equal to or smaller than the proximal surface, in a contact angle with respect to the acoustic matching liquid.

2. The probe array according to claim 1, wherein the reception surface is equal to or higher than the proximal surface, in affinity with the acoustic matching liquid,

3. The probe array according to claim 1, wherein the reception surface is higher than the proximal surface, in a surface tension.

4. The probe array according to claim 1, wherein the reception surface is lower than the proximal surface, in affinity with air.

5. The probe array according to claim 1, wherein in a case where the acoustic matching liquid mainly contains water, a surface that comes in contact with the acoustic matching liquid on the proximal surface is equal to or larger than the reception surface, in a contact angle with respect to pure water.

6. The probe array according to claim 5, wherein the reception surface is equal to or higher than the proximal surface, in affinity with the pure water.

7. The probe array according to claim 5, wherein the reception surface includes metal and an oxide layer of the metal, and the oxide layer is disposed on a side which comes in contact with the acoustic matching liquid.

8. The probe array according to claim 7, wherein the metal contains an element selected from the group consisting of at least aluminum, chrome, zinc, titanium, tantalum, niobium, and zirconium, and the oxide layer is a passive layer of the metal.

9. The probe array according to claim 7, wherein the oxide layer forms water and hydrate contained in the acoustic matching liquid.

10. The probe array according to claim 1, wherein the reception surface and the proximal surface have a metal layer on at least two sides which come in contact with the acoustic matching liquid.

11. The probe array according to claim 10, wherein the reception surface and the proximal surface have an acoustic matching layer configured to have an elastic modulus lower than that of the metal layer and to support the metal layer.

12. The probe array according to claim 11, wherein the acoustic matching layer on the reception surface is smaller in a thickness than the acoustic matching layer on the proximal surface.

13. The probe array according to claim 1,
wherein the plurality of probes each has a paired electrodes which oppose each other across a gap or hold a piezoelectric body therebetween so as to output an acoustic wave signal with signal intensity depending on an electrode area, and
wherein the reception surface is a region which is proximate to a side coming in contact with the acoustic matching liquid and corresponds to the electrode area in one of the paired electrodes.

14. An acoustic wave reception device comprising:
a support base configured to support an examinee; and
a vessel having a vessel portion configured to store acoustic matching liquid at least a coupling liquid level at which the acoustic matching liquid acoustically couples to a subject and a probe array connected to the vessel portion and configured to receive acoustic wave propagated from the subject via the acoustic matching liquid, and
wherein the probe array comprises:
a plurality of probes each having a reception surface which comes in contact with an acoustic matching liquid stored in the vessel; and
a support portion configured to support the plurality of probes, the support portion having a proximal surface which comes in contact with the acoustic matching liquid where the proximal surface is adjacent to the reception surface,
wherein the reception surface shows a contact angle equal to or smaller than the proximal surface, in a contact angle with respect to the acoustic matching liquid.

15. The acoustic wave reception device according to claim 14, wherein the vessel has an opening where the acoustic matching liquid comes in contact with air and is connected to a liquid supply system which supplies the acoustic matching liquid.

16. The acoustic wave reception device according to claim 14,
wherein the probe array is secured to the vessel, and
wherein the vessel is connected to a scanning unit configured to scan the probe array so as to change a position relative to the subject.

17. The acoustic wave reception device according to claim 14, wherein the vessel further includes a temperature control mechanism configured to change a temperature of the acoustic matching liquid stored in the vessel.

18. The acoustic wave reception device according to claim 14, wherein the vessel has an irradiation unit configured to be optically connected to a light source for generating a near infrared ray and to irradiate the subject with the near infrared ray.

19. An acoustic wave reception device comprising:
a support base configured to support an examinee; and
a vessel configured to store the acoustic matching liquid to a coupling liquid level of acoustic coupling to a subject includes the probe array according to claim 1 and an irradiation unit, wherein the irradiation unit is configured to be optically connected to a light source for generating a near infrared ray and to irradiate the subject with the near infrared ray,
wherein the metal layer is a reflection layer configured to reflect the near infrared ray.

20. The acoustic wave reception device according to claim 19, wherein the reflection layer reflects the near infrared ray to the acoustic matching liquid so that the electrodes or the acoustic matching layer do not generate a photoacoustic wave.
